SEQUENCE LISTING (13 sequences)

SEQ ID NO: 1 - DNA, 41 bp, Artificial Sequence
  Oligonucleotide primer
attattgagctctaaggagatatcatatgcgcggcatgctg

SEQ ID NO: 2 - DNA, 36 bp, Artificial Sequence
  Oligonucleotide primer
aataataagctgtacagccgtcttctccccgatgcc

SEQ ID NO: 3 - DNA, 39 bp, Artificial Sequence
  Oligonucleotide primer
gtgcgcctggacgtggaatccctccgggccttgtccctg

SEQ ID NO: 4 - DNA, 36 bp, Artificial Sequence
  Oligonucleotide primer
atatattaagcttcactccttggcggagagccagtc

SEQ ID NO: 5 - protein, 29 aa, Artificial Sequence
  Junction of 3′-5′-exonuclease and polymerase domains of pTne 86 construct
KGIGEKTAVQLLGGVYVDTEFLRALSLEV

SEQ ID NO: 6 - DNA, 27 bp, Artificial Sequence
  Oligonucleotide primer
aagacggctgtacagcttctcggcaag

SEQ ID NO: 7 - DNA, 36 bp, Artificial Sequence
  Oligonucleotide primer
gagcttcatcgatagtatcttgtagagcctataagt

SEQ ID NO: 8 - DNA, 51 bp, Artificial Sequence
  Oligonucleotide primer
atactatcgatgaagctccatgaagagaggctcctttggctttaccgggag

SEQ ID NO: 9 - protein, 14 aa, Artificial Sequence
  Junction of 3′-5′-exonuclease and polymerase domains of pTne 173 construct
LSMKLHEERLLWLY

SEQ ID NO: 10 - protein, 12 aa, Artificial Sequence
  Junction of 3′-5′-exonuclease and polymerase domains of pTne 87 construct
LSMRLEGEERLL

SEQ ID NO: 11 - protein, 11 aa, Artificial Sequence
  Junction of 3′-5′-exonuclease and polymerase domains of pTne 90 construct
RIHASFNQTAT

SEQ ID NO: 12 - DNA, 36 bp, Artificial Sequence
  Oligonucleotide primer
gctccgcgacggcagccacggcgtcggccggcggtt

SEQ ID NO: 13 - DNA, 63 bp, Artificial Sequence
  Oligonucleotide primer
cgaggcgctgccgtcggtgccgcagccggccggtttctgctacgccggtaggctaacgttacg

(19) United States
(12) Patent Application Publication
(10) Pub. No.: US 2002/0119461 A1
(43) Pub. Date: Aug. 29, 2002

(54) High fidelity polymerases and uses thereof

(75) Inventor: Deb K. Chatterjee, North Potomac, MD (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Invitrogen Corporation

(21) Appl. No.: 09/902,741

(22) Filed: Jul. 12, 2001

Related U.S. Application Data
(60) Provisional application No. 60/217,738, filed on Jul. 12, 2000.

(51) Int. Cl. C12Q 1/68; C12P 19/34; C12N 9/22

(52) U.S. Cl. 435/6; 435/91.2; 435/199

(57) ABSTRACT

The invention relates to a DNA and RNA polymerases which have increased fidelity (or reduced misincorporation rate). In particular, the invention relates to a method of making such polymerases by increasing or enhancing 3′-5′ exonuclease activity of a polymerase by, for example, substituting the 3′-5′ exonuclease domain of one polymerase with a 3′-5′ exonuclease domain with the desired activity from another polymerase. The invention also relates to DNA molecules containing the genes encoding the polymerases of the invention, to host cells containing such DNA molecules and to methods to make the polymerases using such host cells. The polymerases of the invention are particularly suited for nucleic acid synthesis, sequencing, amplification and cDNA synthesis.

### BACKGROUND OF THE INVENTION

1. Field of the Invention

The present invention relates to substantially pure polymerases having high fidelity. Specifically, the polymerases of the present invention are polymerases (e.g., DNA polymerases or RNA polymerases) which have been modified to increase the fidelity of the polymerase (compared to the unmodified or unmutated polymerase), thereby providing a polymerase which has a lower misincorporation rate (reduced misincorporation). Preferably, the polymerases of the invention are thermostable or mesophilic polymerases. The present invention also relates to cloning and expression of the polymerases of the invention, to DNA molecules containing the cloned gene, and to hosts which express said genes. The polymerases of the present invention may be used in DNA sequencing, amplification reactions, nucleic acid synthesis and cDNA synthesis.

This invention also relates to polymerases of the invention which have one or more additional mutations or modifications. Such mutations or modifications include those which (1) enhance or increase the ability of the polymerase to incorporate dideoxynucleotides and other modified nucloetides into a DNA molecule about as efficiently as deoxynucleotides; and (2) substantially reduce 5′-3′ exonuclease activity. The polymerases of this invention can have one or more of these properties. These polymerases may also be used in DNA sequencing, amplification reactions, nucleic acid synthesis and cDNA synthesis.

2. Related Art

DNA polymerases synthesize the formation of DNA molecules which are complementary to a DNA template. Upon hybridization of a primer to the single-stranded DNA template, polymerases synthesize DNA in the 5′ to 3′ direction, successively adding nucleotides to the 3′-hydroxyl group of the growing strand. Thus, in the presence of deoxyribonucleoside triphosphates (dNTPs) and a primer, a new DNA molecule, complementary to the single stranded DNA template, can be synthesized.

A number of DNA polymerases have been isolated from mesophilic microorganisms such as E. coli. A number of these mesophilic DNA polymerases have also been cloned. Lin et al. cloned and expressed T4 DNA polymerase in E. coli (Proc. Natl. Acad. Sci. USA 84:7000-7004 (1987)). Tabor et al. (U.S. Pat. No. 4,795,699) describes a cloned T7 DNA polymerase, while Minkley et al. (J. Biol. Chem. 259:10386-10392 (1984)) and Chatterjee (U.S. Pat. No. 5,047,342) described E. coli DNA polymerase I and the cloning of T5 DNA polymerase, respectively.

DNA polymerases from thermophiles have also been described. Chien et al., J. Bacteriol. 127:1550-1557 (1976) describe a purification scheme for obtaining a polymerase from Thermus aquaticus (Taq). The resulting protein had a molecular weight of about 63,000 daltons by gel filtration analysis and 68,000 daltons by sucrose gradient centrifugation. Kaledin et al., Biokhymiya 45:644-51 (1980) disclosed a purification procedure for isolating DNA polymerase from T. aquaticus YT1 strain. The purified enzyme was reported to be a 62,000 dalton monomeric protein. Gelfand et al. (U.S. Pat. No. 4,889,818) cloned a gene encoding a thermostable DNA polymerase from Thermus aquaticus. The molecular weight of this protein was found to be about 86,000 to 90,000 daltons. Simpson et al. purified and partially characterized a thermostable DNA polymerase from a Thermotoga species (Biochem. Cell. Biol. 86:1292-1296 (1990)). The purified DNA polymerase isolated by Simpson et al. exhibited a molecular weight of 85,000 daltons as determined by SDS-polyacrylamide gel electrophoresis and size-exclusion chromatography. The enzyme exhibited half-lives of 3 minutes at 95° C. and 60 minutes at 50° C. in the absence of substrate and its pH optimum was in the range of pH 7.5 to 8.0. Triton X-100 appeared to enhance the thermostability of this enzyme. The strain used to obtain the thermostable DNA polymerase described by Simpson et al. was Thermotoga species strain FjSS3-B.1 (Hussar et al., FEMS Microbiology Letters 37:121-127 (1986)). Others have cloned and sequenced a thermostable DNA polymerase from Thermotoga maritima (U.S. Pat. No. 5,374,553, which is expressly incorporated herein by reference).

Other DNA polymerases have been isolated from thermophilic bacteria including Bacillus steraothermophilus (Stenesh et al., Biochim. Biophys. Acta 272:156-166 (1972); and Kaboev et al., J. Bacteriol. 145:21-26 (1981)) and several archaebacterial species (Rossi et al., System. Appl. Microbiol. 7:337-341 (1986); Klimczak et aL, Biochemistry 25:4850-4855 (1986); and Elie et al., Eur. J. Biochem. 178:619-626 (1989)). The most extensively purified archaebacterial DNA polymerase had a reported half-life of 15 minutes at 87° C. (Elie et al. (1989), supra). Innis et al., In PCR Protocol: A Guide To Methods and Amplification, Academic Press, Inc., San Diego (1990) noted that there are several extreme thermophilic eubacteria and archaebacteria that are capable of growth at very high temperatures (Bergquist et al., Biotech. Genet. Eng Rev. 5:199-244 (1987); and Kelly et al., Biotechnol. Prog. 4:47-62 (1988)) and suggested that these organisms may contain very thermostable DNA polymerases.

In many of the known polymerases, three domains exist, one having the 5′-3′ exonuclease activity, one having the 3′-5′ exonuclease activity, and a third domain which has polymerase activity.

The 5′-3′ exonuclease domain is present in the N-terminal region of the polymerase. (Ollis, et al., Nature 313:762-766 (1985); Freemont et al., Proteins 1:66-73 (1986); Joyce, Cur. Opin. Struct. Biol. 1:123-129 (1991).) There are some amino acids, the mutation of which are thought to impair the 5′-3′ exonuclease activity of E. coli DNA polymerase I. (Gutman & Minton, Nucl. Acids Res. 21:4406-4407 (1993).) These amino acids include Tyr⁷⁷, Gly¹⁰³, Gly¹⁸⁴, and Gly¹⁹² in E. coli DNA polymerase I. It is known that the 5′-exonuclease domain is dispensable. The best known example is the Klenow fragment of E. coli polymerase I. The Klenow fragment is a natural proteolytic fragment devoid of 5′-exonuclease activity (Joyce et. al., J. Biol. Chem. 257:1958-64 (1990).) Polymerases lacking this activity are useful for DNA sequencing.

The polymerase active site, including the dNTP binding domain is usually present at the carboxyl terminal region of the polymerase (Ollis et al., Nature 313:762-766 (1985); Freemont et al., Proteins 1:66-73 (1986)). It has been shown that Phe⁷⁶² of E. coli polymerase I is one of the amino acids that directly interacts with the nucleotides (Joyce & Steitz, Ann. Rev. Biochem. 63:777-822 (1994); Astatke, J. Biol. Chem. 270:1945-54 (1995)). Converting this amino acid to a Tyr results in a mutant DNA polymerase that does not discriminate against dideoxynucleotides. See U.S. Pat. Nos. 5,614,365 5,912,155, 5,939,301, 6,015,668 and 5,948,614, and copending U.S. application Ser. No. 08/525,057, of Deb K. Chatterjee, filed Sep. 8, 1995, entitled “Mutant DNA Polymerases and the Use Thereof,” which is expressly incorporated herein by reference.

Most DNA polymerases also contain a 3′-5′ exonuclease activity. This exonuclease activity provides a proofreading ability to the DNA polymerase. Taq DNA polymerase from Thermus aquaticus, the most user friendly in nucleic acid synthesis reactions, hence most popular enzyme for use in polymerase chain reactions (PCR), does not have proofreading ability. In comparison with other enzymes, the relative average error rates for Taq compared to polymerases such as Pfu, Vent and Deep Vent polymerases which do have proofreading capability were estimated to be 8×10⁻⁶, 1.3×10⁻⁶, 2.8×10⁻⁶ and 2.7×10⁻⁶ respectively (Cline et. al., Nucleic Acids Res. 24:3546-3551(1996)). This is due to the fact that Taq DNA polymerase has deletions in all three important motifs required for 3′-5′ exonuclease activity (Lawyer et al., J. Biol. Chem. 6427-6437 (1989)). Interestingly, even with the deletions, Taq DNA polymerase maintains the overall three dimensional structure compared to Klenow fragment albeit dramatically altered in the vestigial 3′-5′ exonuclease domain (Kim et al., Nature 376:612-616 (1995); Eom et al., Nature 382:278-281(1996)).

While polymerases are known, there exists a need in the art to develop polymerases which are more suitable for nucleic acid synthesis, sequencing, and amplification. Such polymerases would have reduced error rate; that is reduced misincorporation of nucleotides during nucleic acid synthesis and/or increased fidelity of polymerization.

### SUMMARY OF THE INVENTION

The present invention satisfies these needs in the art by providing additional polymerases useful in molecular biology. Specifically, this invention includes thermostable and mesophilic polymerases which have increased fidelity. Such polymerases are modified in their 3′-5′ exonuclease domain such that the fidelity of the enzyme is increased or enhanced.

Modifications can include mutations in the 3′-5′ exonuclease domain which result in increased 3′-5′ exonuclease activity, or partial or complete substitution of the 3′-5′ exonuclease domain with a 3′-5′ exonuclease domain from a polymerase having increased 3′-5′ exonuclease activity.

In the present invention, we have made hybrid Taq polymerase where the inactive 3′-5′-exonuclease domain of Taq polymerase was replaced with an active 3′-5′-exonuclease domain from another thermostable DNA polymerase. We have recently reported a thermostable DNA polymerase from Thermotoga neapolitana, Tne DNA polymerase (U.S. Pat. Nos. 5,912,155, 5,939,301, 6,015,668 and 5,948,614). Similar to Taq polymerase, the Tne polymerase also belongs to the Pol I family. However, unlike Taq polymerase, Tne polymerase has an active 3′-5′-exonuclease domain. We have shown that the hybrid Taq polymerase displayed all three activities, 5′-3′-exonuclease activity, 3′-5′-exonuclease activity and the polymerase activity suggesting that the domain shuffling did not impair the structural integrity. We have also shown that both proof-reading activity and the polymerase act in concert indicating that the hybrid polymerase is acting like a true high-fidelity polymerase. Therefore, the hybrid polymerase will be extremely useful for PCR or other applications.

DNA polymerases (including thermostable DNA polymerases) of particular interest in the invention include Taq DNA polymerase, Tne DNA polymerase, Tma DNA polymerase, Pfu DNA polymerase, Tfl DNA polymerase, Tth DNA polymerase, Tbr DNA polymerase, Pwo DNA polymerase, Bst DNA polymerase, Bca DNA polymerase, VENT™ DNA polymerase, T7 DNA polymerase, T5 DNA polymerase, DNA polymerase III, Klenow fragment DNA polymerase, Stoffel fragment DNA polymerase, and mutants, fragments or derivatives thereof In accordance with the invention, such polymerase are modified or mutated in the 3′-5′ exonuclease domain so as to increase fidelity of the enzyme of interest.

The present invention relates in particular to mutant PolI type DNA polymerase (preferably thermostable DNA polymerases) wherein one or more amino acid changes have been made in the 3′-5′ exonuclease domain which renders the enzyme more faithful (higher fidelity) in nucleic acid synthesis, sequencing and amplification. The 3′-5′ exonuclease domain is defined as the region that contains all of the catalytic amino acids (Derbyshire et al., Methods in Enzymology 262:363-385 (1995); Blanco et al., Gene 112:139-144 (1992)). In particular, the three subdomains are Exo I, ExoII and Exo III for DNA polymerases. Exo I for pol I type DNA polymerases is defined by the region 350P to 360S, for Exo II 416K to 429A, and for Exo III 492E to 505T.

Corresponding regions are also found in other DNA polymerases. All three sudomains in the 3′-5′ exo domain should be present for full 3′-5′ activity.

One can modulate according to the invention the exo activity by mutation of specific amino acids or regions in these subdomains using techniques well known in the art.

In accordance with the invention, other functional changes may be made to the polymerases having increased fidelity. For example, the polymerase may also be modified to reduce 5′ exonuclease activity, and/or reduce discrimination against ddNTP's.

In particular, the invention relates to mutant or modified DNA polymerases which are modified in at least one way selected from the group consisting of

(a) to increase the 3 ′-5′ exonuclease activity of the polymerase;

(b) to reduce or eliminate the 5′-3′ exonuclease activity of the polymerase;

(c) to reduce or eliminate discriminatory behavior against dideoxynucleotides or modified nucleotides, and

(d) to reduce or eliminate misincorporation of incorrect nucleotides during nucleic acid synthesis.

The present invention is also directed to DNA molecules (preferably vectors) containing a gene encoding the mutant or modified polymerases of the present invention and to host cells containing such DNA molecules. Any number of hosts may be used to express the gene of interest, including prokaryotic and eukaryotic cells. Preferably, prokaryotic cells are used to express the polymerases of the invention. The preferred prokaryotic host according to the present invention is E. coli.

The invention also relates to a method of producing the polymerases of the invention, said method comprising:

(a) culturing the host cell comprising a gene encoding the polymerases of the invention;

(b) expressing said gene; and

(c) isolating said polymerase from said host cell.

The invention also relates to a method of synthesizing a nucleic acid molecule comprising:

(a) mixing a nucleic acid template (e.g. RNA or DNA) with one or more polymerases of the invention; and

(b) incubating said mixture under conditions sufficient to synthesize a nucleic acid molecule complementary to all or a portion of said template. Such condition may include incubation with one or more deoxy- or dideoxyribonucleoside triphosphates. Such deoxy- and dideoxyribonucleoside triphosphates include dATP, dCTP, dGTP, dTTP, dITP, 7-deaza-dGTP, 7-deaza-dATP, dUTP, ddATP, ddCTP, ddGTP, ddlTP, ddTTP, [a-S]dATP, [α-S]dTTP, [α-S]dGTP, and [α-S]dCTP.

The invention also relates to a method of sequencing a DNA molecule, comprising:

(a) hybridizing a primer to a first DNA molecule;

(b) contacting said molecule of step (a) with deoxyribonucleoside triphosphates, one or more DNA polymerases of the invention, and one or more terminator nucleotides;

(c) incubating the mixture of step (b) under conditions sufficient to synthesize a random population of DNA molecules complementary to said first DNA molecule, wherein said synthesized DNA molecules are shorter in length than said first DNA molecule and wherein said synthesized DNA molecules comprise a terminator nucleotide at their 3′ termini; and

(d) separating said synthesized DNA molecules by size so that at least a part of the nucleotide sequence of said first DNA molecule can be determined. Such terminator nucleotides include ddTTP, ddATP, ddGTP, ddITP or ddCTP.

The invention also relates to a method for amplifying a double stranded DNA molecule, comprising:

(a) providing a first and second primer, wherein said first primer is complementary to a sequence within or at or near the 3 ′-termini of the first strand of said DNA molecule and said second primer is complementary to a sequence within or at or near the 3′-termini of the second strand of said DNA molecule;

(b) hybridizing said first primer to said first strand and said second primer to said second strand in the presence of one or more polymerases of the invention, under conditions such that a third DNA molecule complementary to all or a portion of said first strand and a fourth DNA molecule complementary to all or a portion of said second strand are synthesized;

(c) denaturing said first and third strand, and said second and fourth strands; and

(d) repeating steps (a) to (c) one or more times.

Thus, the invention generally relates to amplifying or sequencing nucleic acid molecules comprising:

(a) mixing one or more templates or nucleic acid molecules to be sequenced with one or more of the polymerases of the invention and

(b) incubating said mixture under conditions sufficient to amplify all or a portion of said templates or sequence all or a portion of said nucleic acid molecules.

The invention also relates to a kit for sequencing, amplifying or synthesis of a nucleic acid molecule comprising one or more polymerases of the invention and one or more other components (or combinations thereof) selected from the group consisting of

(a) one or more dideoxyribonucleoside triphosphates;

(b) one or more deoxyribonucleoside triphosphates;

(c) one or more primers;

(d) one or more suitable buffers or buffering salts;

(e) one or more nucleotides; and

(f) instructions for carrying out the methods of the invention.

The invention also relates to compositions made for carrying out the methods of the invention and compositions made while carrying out the methods of the invention. Such compositions may comprise one or more components selected from the group consisting of one or more polymerases of the invention, one or more nucleotides, one or more templates, one or more reaction buffers or buffering salts, one or more primers, one or more nucleic acid products made by the methods of the invention and the like.

### BRIEF DESCRIPTION OF THE DRAWINGS/FIGURES

FIG. 1 depicts gels showing the relative 3′-5′ exonuclease activity of Tne DNA polymerase and mutant derivatives determined qualitatively using a 36/64mer primer template substrate, that has a four base mismatch at the 3′ terminus of the primer strand, at 60° C. TneA denotes a Tne DNA polymerase mutant that carries D137A and D323A (deficient in the 5′-3′ exonuclease and 3′-5′ exonuclease activities); TneB denotes a Tne DNA polymerase mutant that carries D137A, deficient in the 5′-3′ exonuclease activity; Chi denotes a Taq/Tne chimeric DNA polymerase as described below and Taq is the wild-type Taq DNA polymerase. The three lanes, of each panel, from left to right are 20 sec, 1 min, and 2 min, time points that have elapsed before the reactions were quenched. P denotes the primer position, and C (2 lanes) is the control in which no enzyme was added to the reaction mix.

FIG. 2 depicts gels showing the ability of Tne DNA polymerase and mutant derivatives to degrade a mismatch from the primer termini and initiate the incorporation of dNTP determined qualitatively using a 36/64mer primer template substrate, that has a four base mismatch at the 3'terminus of the primer strand, at 60° C. TneA denote a Tne DNA polymerase mutant that carries D137A and D323A, deficient in the 5′-3′ exonuclease and 3′-5′ exonuclease activities; TneB denote a Tne DNA polymerase mutant that carries D137A, deficient in the 5′-3′ exonuclease activity; Taq is the wild-type Taq DNA polymerase and Chi denotes a Tne-Taq chimeric DNA polymerase as described below. The four lanes, of each panel, from left to right are 20 sec, 1 min, 2 min and 5 min, time points that have elapsed before the reactions were quenched. P denotes the primer position, and C (2 lanes) is the control in which no enzyme was added to the reaction mix.

### DETAILED DESCRIPTION OF THE INVENTION

Definitions

In the description that follows, a number of terms used in recombinant DNA technology are extensively utilized. In order to provide a clearer and consistent understanding of the specification and claims, including the scope to be given such terms, the following definitions are provided.

Cloning vector. A plasmid, cosmid or phage DNA or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be spliced in order to bring about its replication and cloning. The cloning vector may further contain a marker suitable for use in the identification of cells transformed with the cloning vector. Markers, for example, are tetracycline resistance or ampicillin resistance.

Expression vector. A vector similar to a cloning vector but which is capable of enhancing the expression of a gene which has been cloned into it, after transformation into a host. The cloned gene is usually placed under the control of (i.e., operably linked to) certain control sequences such as promoter sequences.

Recombinant host. Any prokaryotic or eukaryotic or microorganism which contains the desired cloned genes in an expression vector, cloning vector or any DNA molecule. The term “recombinant host” is also meant to include those host cells which have been genetically engineered to contain the desired gene on the host chromosome or genome.

Host. Any prokaryotic or eukaryotic microorganism that is the recipient of a replicable expression vector, cloning vector or any DNA molecule. The DNA molecule may contain, but is not limited to, a structural gene, a promoter and/or an origin of replication.

Promoter. A DNA sequence generally described as the 5′ region of a gene, located proximal to the start codon. At the promoter region, transcription of an adjacent gene(s) is initiated.

Gene. A DNA sequence that contains information necessary for expression of a polypeptide or protein. It includes the promoter and the structural gene as well as other sequences involved in expression of the protein.

Structural gene. A DNA sequence that is transcribed into messenger RNA that is then translated into a sequence of amino acids characteristic of a specific polypeptide.

Operably linked. As used herein means that the promoter is positioned to control the initiation of expression of the polypeptide encoded by the structural gene.

Expression. Expression is the process by which a gene produces a polypeptide. It includes transcription of the gene into messenger RNA (mRNA) and the translation of such mRNA into polypeptide(s).

Substantially Pure. As used herein “substantially pure” means that the desired purified protein is essentially free from contaminating cellular contaminants which are associated with the desired protein in nature.

Contaminating cellular components may include, but are not limited to, phosphatases, exonucleases, endonucleases or undesirable DNA polymerase enzymes.

Primer. As used herein “primer” refers to a single-stranded oligonucleotide that is extended by covalent bonding of nucleotide monomers during amplification or polymerization of a DNA molecule.

Template. The term “template” as used herein refers to a double-stranded or single-stranded nucleic acid (DNA or RNA such as mRNA) molecule which is to be amplified, synthesized or sequenced. In the case of a double-stranded nucleic acid molecule, denaturation of its strands to form a first and a second strand is performed before these molecules may be amplified, synthesized or sequenced. A primer, complementary to a portion of a template is hybridized under appropriate conditions and the polymerase of the invention may then synthesize a molecule complementary to said template or a portion thereof. The newly synthesized molecule, according to the invention, may be equal or shorter in length than the original template.

Additionally, the newly synthesized nucleic acid molecules may serve as templates for further synthesis according to the invention. Mismatch incorporation during the synthesis or extension of the newly synthesized molecule may result in one or a number of mismatched base pairs. Thus, the synthesized molecule need not be exactly complementary to the template.

Incorporating. The term “incorporating” as used herein means becoming a part of a DNA molecule or primer.

Amplification. As used herein “amplification” refers to any in vitro method for increasing the number of copies of a nucleotide sequence with the use of a DNA polymerase. Nucleic acid amplification results in the incorporation of nucleotides into a DNA molecule or primer thereby forming a new DNA molecule complementary to a DNA template. The formed DNA molecule and its template can be used as templates to synthesize additional DNA molecules. As used herein, one amplification reaction may consist of many rounds of DNA replication. DNA amplification reactions include, for example, polymerase chain reactions (PCR). One PCR reaction may consist of 20 to 100 “cycles” of denaturation and synthesis of a DNA molecule.

Oligonucleotide. “Oligonucleotide” refers to a synthetic or natural molecule comprising a covalently linked sequence of nucleotides which are joined by a phosphodiester bond between the 3′ position of the pentose of one nucleotide and the 5′ position of the pentose of the adjacent nucleotide.

Nucleotide. As used herein “nucleotide” refers to a base-sugar-phosphate combination. Nucleotides are monomeric units of a nucleic acid sequence (DNA and RNA). The term nucleotide includes deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Thermostable. As used herein “thermostable” refers to a DNA polymerase which is resistant to inactivation by heat. DNA polymerases synthesize the formation of a DNA molecule complementary to a single-stranded DNA template by extending a primer in the 5′-to-3′ direction. This activity for mesophilic DNA polymerases may be inactivated by heat treatment. For example, T5 DNA polymerase activity is totally inactivated by exposing the enzyme to a temperature of 90° C. for 30 seconds. As used herein, a thermostable DNA polymerase activity is more resistant to heat inactivation than a mesophilic DNA polymerase. However, a thermostable DNA polymerase does not mean to refer to an enzyme which is totally resistant to heat inactivation and thus heat treatment may reduce the DNA polymerase activity to some extent. A thermostable DNA polymerase typically will also have a higher optimum temperature than mesophilic DNA polymerases.

Hybridization. The terms “hybridization” and “hybridizing” refers to the pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used.

3′-to-5′ Exonuclease Activity. “3′-to-5′ exonuclease activity” is an enzymatic activity well known to the art. This activity is often associated with DNA polymerases, and is thought to be involved in a DNA replication “editing” or correction mechanism.

A “DNA polymerase increased in 3′-to-5′ exonuclease activity” is defined herein as a mutated DNA polymerase that has about or more than 10% increase, or preferably about or more than 25%, 30%, 50%, 100%, 150%, 200%, or 300% increase in the 3′-to-5′ exonuclease activity compared to the corresponding unmutated, wild-type enzyme. An increase in 3′-5′ exonuclease activity for a polymerase of the invention may also be measured according to relative activity compared to the corresponding unmodified or wild type polymerase. Preferably, the increase in such relative activity is 1.5, 2, 5, 10, 25, 50, 75, 100, 150, 200, or 300 fold comparing the activity of the 3′-5′ exonuclease activity of the polymerase of the invention to its corresponding unmutated or unmodified enzyme. Alternatively, the 3′-5′ exonuclease activity of the polymerase of the invention may be measured directly as specific activity which may range from about 0.005, 0.01, 0.05, 0.75, 0.1, 0.15, 0.4, 0.5, 0.75, 0.9, 1.0, 1.2, 1.5, 1.75, 2.0, 3.0, 5.0, 7.5, 10, 15, 20, 30 unit/mg protein. A unit of activity of 3′-to-5′ exonuclease is defined as the amount of activity that solubilizes 10 nmoles of substrate ends in 60 min at 37° C., assayed as described in the “BRL 1989 Catalogue & Reference Guide,” page 5, with HhaI fragments of lambda DNA 3′-end labeled with [H]dTTP by terminal deoxynucleotidyl transferase (TdT). Protein is measured by the method of Bradford, Anal. Biochem. 72:248 (1976). As a means of comparison, natural, wild-type T5-DNA polymerase (DNAP) or T5-DNAP encoded by pTTQ19-T5-2 has a specific activity of about 10 units/mg protein while the DNA polymerase encoded by pTTQ19-T5-2(Exo⁻) (U.S. Pat. 5,270,179) has a specific activity of about 0.0001 units/mg protein, or 0.001% of the specific activity of the unmodified enzyme, a 10⁵-fold reduction.

5′-to-3′ Exonuclease Activity. “5′-to-3′ exonuclease activity” is also an enzymatic activity well known in the art. This activity is often associated with DNA polymerases, such as E. coli PolI and PolIII.

A “DNA polymerase substantially reduced in 5′-to-3′ exonuclease activity” is defined herein as either (1) a mutated DNA polymerase that has about or less than 10%, or preferably about or less than 1%, of the 5′-to-3′ exonuclease activity of the corresponding unmutated, wild-type enzyme, or (2) a DNA polymerase having 5′-to-3′ exonuclease specific activity which is less than about 1 unit/mg protein, or preferably about or less than 0.1 units/mg protein.

Both of the 3′-to-5′ and 5′-to-3′ exonuclease activities can be observed on sequencing gels. Active 5′-to-3′ exonuclease activity will produce nonspecific ladders in a sequencing gel by removing nucleotides from the 5′-end of the growing primers. 3′-to-5′ exonuclease activity can be measured by following the degradation of radiolabeled primers in a sequencing gel. Thus, the relative amounts of these activities, e.g. by comparing wild-type and mutant polymerases, can be determined with no more than routine experimentation.

Fidelity. Fidelity refers to the accuracy of polymerization, or the ability of the polymerase to discriminate correct from incorrect substrates, (e.g., nucleotides) when synthesizing nucleic acid molecules (e.g. RNA or DNA) which are complementary to a template. The higher the fidelity of a polymerase, the less the polymerase misincorporates nucleotides in the growing strand during nucleic acid synthesis; that is, an increase or enhancement in fidelity results in a more faithful polymerase having decreased error rate (decreased misincorporation rate).

A DNA polymerase having increased/enhanced/higher fidelity is defined as a polymerase having about 2 to about 10,000 fold, about 2 to about 5,000 fold, or about 2 to about 2000 fold (preferably greater than about 5 fold, more preferably greater than about 10 fold, still more preferably greater than about 50 fold, still more preferably greater than about 100 fold, still more preferably greater than about 500 fold and most preferably greater than about 1000 fold) reduction in the number of misincorporated nucleotides during synthesis of any given nucleic acid molecule of a given length. For example, a mutated polymerase may misincorporate one nucleotide in the synthesis of 1000 bases compared to an unmutated polymerase miscincorporating 10 nucleotides. Such a mutant polymerase would be said to have an increase of fidelity of 10 fold.

A DNA polymerase having reduced misincorporation is defined herein as either a mutated or modified DNA polymerase that has about or less than 50%, or preferably about or less than 25%, more preferably about or less than 10% and most preferably about or less than 1% of relative misincorporation compared to the corresponding unmutated, unmodified or wild type enzyme. A less fidelity DNA polymerase may also initiate DNA synthesis with an incorrect nucleotide incorporation (Perrion & Loeb, 1989, J. Biol. Chem. 264:2898-2905).

The fidelity or misincorporation rate of a polymerase can be determined by sequencing or by other method known in the art (Eckert & Kunkel, Nucl. Acids Res. 3739-3744(1990)). In one example, the sequence of a DNA molecule synthesized by the unmutated and mutated polymerase can be compared to the expected (known) sequence. In this way, the number of errors (misincorporation) can be determined for each enzyme and compared.

In another example, the unmutated and mutated polymerase may be used to sequence a DNA molecule having a known sequence. The number of sequencing errors (misincorporation) can be compared to determine the fidelity or misincorporation rate of the enzymes. Other means of determining the fidelity or misincorporation rate will be recognized by one of skill in the art.

1. Sources of Polymerases

A variety of polypeptides having polymerase activity are useful in accordance with the present invention. Included among these polypeptides are enzymes such as nucleic acid polymerases (including DNA polymerases).

Such polymerases include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™) DNA polymerase, Pyrococcus furiosus (Pfu) DNA polymerase, DEEPVEN™ DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, mycobacterium DNA polymerase (Mtb, Mlep), and mutants, and variants and derivatives thereof.

Polymerases used in accordance with the invention may be any enzyme that can synthesize a nucleic acid molecule from a nucleic acid template, typically in the 5′ to 3′ direction. The nucleic acid polymerases used in the present invention may be mesophilic or thermophilic, and are preferably thermophilic. Preferred mesophilic DNA polymerases include T7 DNA polymerase, T5 DNA polymerase, Klenow fragment DNA polymerase, DNA polymerase III and the like. Preferred thermostable DNA polymerases that may be used in the methods of the invention include Taq, Tne, Tma, Pfu, Tfl, Tth, Stoffel fragment, VENT™ and DEEPVENT™ DNA polymerases, and mutants, variants and derivatives thereof (U.S. Pat. No. 5,436,149; U.S. Patent 4,889,818; U.S. Pat. No. 4,965,188; U.S. Pat. No. 5,079,352; U.S. Patent 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J. -M, et al., Nuc. Acids Res. 22(15):3259-3260 (1994)). For amplification of long nucleic acid molecules (e.g., nucleic acid molecules longer than about 3-5 Kb in length), at least two DNA polymerases (one substantially lacking 3′ exonuclease activity and the other having 3′ exonuclease activity) are typically used. See U.S. Pat. No. 5,436,149; U.S. Pat. No. 5,512,462; Fames, W. M., Gene 112:29-35 (1992); and copending U.S. patent application Ser. No. 09/741,664, filed Dec. 21, 2000, the disclosures of which are incorporated herein in their entireties. Examples of DNA polymerases substantially lacking in 3′ exonuclease activity include, but are not limited to, Taq, Tne(exo⁻), Tma(exo⁻), Pfu(exo⁻), Pwo(exo⁻) and Tth DNA polymerases, and mutants, variants and derivatives thereof.

Polypeptides having nucleic acid polymerase activity are preferably used in the present methods at a final concentration in solution of about 0.1-200 units per milliliter, about 0.1-50 units per milliliter, about 0.1-40 units per milliliter, about 0.1-3.6 units per milliliter, about 0.1-34 units per milliliter, about 0.1-32 units per milliliter, about 0.1-30 units per milliliter, or about 0.1-20 units per milliliter, and most preferably at a concentration of about 20 units per milliliter. Of course, other suitable concentrations of nucleic acid polymerases suitable for use in the invention will be apparent to one or ordinary skill in the art.

In a preferred aspect of the invention, mutant or modified polymerases are made by recombinant techniques. A number of cloned polymerase genes are available or may be obtained using standard recombinant techniques.

To clone a gene encoding a DNA polymerase which will be modified in accordance with the invention, isolated DNA which contains the polymerase gene is used to construct a recombinant DNA library in a vector.

Any vector, well known in the art, can be used to clone the DNA polymerase of interest. However, the vector used must be compatible with the host in which the recombinant DNA library will be transformed.

Prokaryotic vectors for constructing the plasmid library include plasmids such as those capable of replication in E. coli such as, for example, pBR322, ColE1, pSC101, pUC-vectors (pUC18, pUC19, etc.: In: Molecular Cloning, A Laboratory Manual, Cold Spring Harbor Laboratory Press, Cold Spring Harbor, N.Y. (1982); and Sambrook et al., In: Molecular Cloning A Laboratory Manual (2d ed.) Cold Spring Harbor Laboratory Press, Cold Spring Harbor, N.Y. (1989)). Bacillus plasmids include pC194, pC221, pC217, etc. Such plasmids are disclosed by Glyczan, T. In: The Molecular Biology Bacilli, Academic Press, York (1982), 307-329. Suitable Streptomyces plasmids include pIJ101 (Kendall et al., J. Bacteriol 169:4177-4183 (1987)). Pseudomonas plasmids are reviewed by John et al., (Rad Insec. Dis. 8:693-704 (1986)), and Igaki, (Jpn. J. Bacteriol. 33:729-742 (1978)). Broad-host range plasmids or cosmids, such as pCP13 (Darzins and Chakrabarbary, J. Bacteriol. 159:9-18, 1984) can also be used for the present invention. The preferred vectors for cloning the genes of the present invention are prokaryotic vectors. Preferably, pCP13 and pUC vectors are used to clone the genes of the present invention.

The preferred host for cloning the polymerase genes of interest is a prokaryotic host. The most preferred prokaryotic host is E. coli. However, the desired polymerase genes of the present invention may be cloned in other prokaryotic hosts including, but not limited to, Escherichia, Bacillus, Streptomyces, Pseudomonas, Salmonella, Serratia, and Proteus. Bacterial hosts of particular interest include E. coli DH10B, which may be obtained from Invitrogen Corporation, Life Technologies Division (Rockville, Md.).

Eukaryotic hosts for cloning and expression of the polymerases of interest include yeast, fungi, and mammalian cells. Expression of the desired polymerase in such eukaryotic cells may require the use of eukaryotic regulatory regions which include eukaryotic promoters. Cloning and expressing the polymerase gene in eukaryotic cells may be accomplished by well known techniques using well known eukaryotic vector systems.

Once a DNA library has been constructed in a particular vector, an appropriate host is transformed by well known techniques. Transformed colonies are plated at a density of approximately 200-300 colonies per petri dish. For thermostable polymerase selection, colonies are then screened for the expression of a heat stable DNA polymerase by transferring transformed E. coli colonies to nitrocellulose membranes. After the transferred cells are grown on nitrocellulose (approximately 12 hours), the cells are lysed by standard techniques, and the membranes are then treated at 95° C. for 5 minutes to inactivate the endogenous E. coli enzyme. Other temperatures may be used to inactivate the host polymerases depending on the host used and the temperature stability of the polymerase to be cloned. Stable polymerase activity is then detected by assaying for the presence of polymerase activity using well known techniques. Sagner et al., Gene 97:119-123 (1991), which is hereby incorporated by reference in its entirety. The gene encoding a polymerase of the present invention can be cloned using the procedure described by Sagner et al., supra.

2. Modifications or Mutations of Polymerases

In accordance with the invention, the 3′-5′ exonuclease domain of the polymerase of interest is modified or mutated in such a way as to produce a mutated or modified polymerase having increased or enhanced fidelity (decreased misincorporation rate). The 3′-5′ exonuclease domain is composed of three subdomains, exo I, exoII, and exoIII (Blanco et al., Gene 112:139-144 (1992)), in which are found the catalytic amino acids which are important for exonuclease activity. The catalytic amino acids interact with metal ions. When introducing mutations into the exonuclease domain, it is preferred that the catalytic amino acids retain their metal interaction. One or more mutations may be made in the exonuclease domain of any polymerase in order to increase fidelity of the enzyme in accordance with the invention. Such mutations include point mutation, flame shift mutations, deletions and insertions. Preferably, one or more point mutations, resulting in one or more amino acid substitutions, are used to produce polymerases having enhanced or increased fidelity or increased or enhanced 3′-5′ exonuclease activity in accordance with the invention. In a preferred aspect of the invention, one or more mutations may be made to produce the desired result.

3. Substitution of the 3′-5′ Exonuclease Domain

Recruitment of new properties from one enzyme into another related enzyme is an exciting prospect of protein engineering. A traditional approach used to yield new properties entailed random mutagenesis and screening a large number of mutants to isolate a few mutants of interest. Another approach is to incorporate specific domains into a new but related protein or enzyme based on structural information (Review article by Pierre Beguin, Curr. Opin. Biotech. 10:336-340 (1999)).

Using techniques well known in the art (Sambrook et al., (1989) in: Molecular Cloning, A Laboratory Manual (2nd Ed.), Cold Spring Harbor Laboratory Press, Cold Spring Harbor, N.Y.), the 3′-5′ exonuclease domain of a DNA polymerase can be substituted with a 3′-5′ exonuclease domain from another polymerase which has the desired 3′-5′ exonuclease activity. Domains of various polymerases are shown in Table 1.

TABLE 1
Approximate domains of different polymerases

| | 5′-3′ exonuclease | 3′-5′ exonuclease | polymerase |
|---|---|---|---|
| E. coli poll | 1-325 aa | 326-419 aa | 420-929 aa |
| Taq polymerase | 1-289 aa | 294-422 aa | 424-831 aa |
| Tne polymerase | 1-294 aa | 295-485 aa | 486-893 aa |
| Tma polymerase | 1-291 aa | 292-484 aa | 485-893 aa |
| T7 polymerase | | 1-187 aa | 202-698 aa |
| T5 polymerase | | 1-334 aa | 335-855 aa |
| Bst polymerase | 1-301 aa | 302-468 aa | 470-876 aa |

Domain substitution of all or a portion of one domain with a different domain is contemplated by the invention. Any domain (or portion thereof) of one polymerase may be substituted with a domain (or portion thereof) of a second polymerase. Preferably, such substitutions are made so that the substitution results in proper folding of the protein such that the desired 3′-5′ exonuclease activity is produced.

4. Additional Modifications or Mutations of Polymerases

In accordance with the invention, in addition to the mutations described above for creating polymerases with lower misincorporation or for enhancing fidelity, one or more additional mutations or modifications (or combinations thereof) may be made to the polymerases of interest. Mutations or modifications of particular interest include those modifications of mutations which (1) eliminate or reduce 5′ to 3′ exonuclease activity; and (2) reduce discrimination of dideoxynucleotides (that is, increase incorporation of dideoxynucleotides).

The 5′-3′ exonuclease activity of the polymerases can be reduced or eliminated by mutating the polymerase gene or by deleting the 5′ to 3′ exonuclease domain. Such mutations include point mutations, frame shift mutations, deletions, and insertions. Preferably, the region of the gene encoding the 5′-3′ exonuclease activity is deleted using techniques well known in the art. In embodiments of this invention, any one of six conserved amino acids that are associated with the 5′-3′ exonuclease activity can be mutated. Examples of these conserved amino acids with respect to Tne DNA polymerase include Asp⁸, Glu¹¹², Asp¹¹⁴, Asp¹¹⁵, Asp¹³⁷ and Asp¹³⁹. Other possible sites for mutation are: Gly¹⁰², Gly¹⁸⁷ and Gly¹⁹⁵.

Corresponding amino acid to target for other polymerases to reduce or eliminate 5′-3′ exonuclease activity as follows:

E. coli poli: Asp¹³, Glu¹¹³, Asp¹¹⁵, Asp¹¹⁶, Asp¹³⁸, and Asp¹⁴⁰.

Taq pol: Asp¹⁸, Glu¹¹⁷, Asp¹¹⁹, Asp¹²⁰, Asp¹⁴², and Asp¹⁴⁴.

Tma pol: Asp⁸, Glu¹¹², Asp¹¹⁴, Asp¹¹⁵, Asp¹³⁷, and Asp¹³⁹.

Amino acid residues of Taq DNA polymerase are as numbered in U.S. Pat. No. Pat. No. 5,079,352. Amino acid residues of Thermotoga maritima (Tma) DNA polymerase are numbered as in U.S. Pat. No. 5,374,553.

Examples of other amino acids which may be targeted for other polymerases to reduce 5′ to 3′ exonuclease activity

| Enzyme or source | Mutation positions |
|---|---|
| Streptococcus | Asp¹⁰, Glu¹¹⁴, Asp¹¹⁶, Asp¹¹⁷, Asp¹³⁹, Asp¹⁴¹ |
| pneumoniae | |
| Thermus flavus | Asp¹⁷, Glu¹¹⁶, Asp¹¹⁸, Asp¹¹⁹, Asp¹⁴¹, Asp¹⁴³ |
| Thermus thermophilus | Asp¹⁸, Glu¹¹⁸, Asp¹²⁰, Asp¹²¹, Asp¹⁴³, Asp¹⁴⁵ |
| Deinococcus radiodurans | Asp¹⁸, Glu¹¹⁷, Asp¹¹⁹, Asp¹²⁰, Asp¹⁴², Asp¹⁴⁴ |
| Bacillus caldotenax | Asp⁹, Glu¹⁰⁹, Asp¹¹¹, Asp¹¹², Asp¹³⁴, Asp¹³⁶ |

Coordinates of S. pneumoniae, T. flavus, D. radiodurans, B. caldotenax were obtained from Gutman and Minton (Nucleic Acids Res. 21: 4406-4407 (1993)). Coordinates of T. thermophilus were obtained from International Patent Appln. No. WO 92/06200.

Polymerase mutants can also be made to render the polymerase non-discriminating against non-natural nucleotides such as dideoxynucleotides (see U.S. Pat. No. 5,614,365). Changes within the O-helix, such as other point mutations, deletions, and insertions, can be made to render the polymerase non-discriminating. By way of example, one Tne DNA polymerase mutant having this property substitutes a nonnatural amino acid such as Tyr for Phe730 in the 0-helix.

Typically, the 5′-3′ exonuclease activity, 3′ to 5′ exonuclease activity, discriminatory activity and fidelity can be affected by substitution of amino acids typically which have different properties. For example, an acidic amino acid such as Asp may be changed to a basic, neutral or polar but uncharged amino acid such as Lys, Arg, His (basic); Ala, Val, Leu, Ile, Pro, Met, Phe, Trp (neutral); or Gly, Ser, Thr, Cys, Tyr, Asn or Gln (polar but uncharged).

Glu may be changed to Asp, Ala, Val Leu, Ile, Pro, Met, Phe, Trp, Gly, Ser, Thr, Cys, Tyr, Asn or Gln.

Preferably, oligonucleotide directed mutagenesis is used to create the mutant polymerases which allows for all possible classes of base pair changes at any determined site along the encoding DNA molecule. In general, this technique involves annealing a oligonucleotide complementary (except for one or more mismatches) to a single stranded nucleotide sequence coding for the DNA polymerase of interest. The mismatched oligonucleotide is then extended by DNA polymerase, generating a double stranded DNA molecule which contains the desired change in sequence on one strand. The changes in sequence can of course result in the deletion, substitution, or insertion of an amino acid. The double stranded polynucleotide can then be inserted into an appropriate expression vector, and a mutant polypeptide can thus be produced. The above-described oligonucleotide directed mutagenesis can of course be carried out via PCR.

5. Enhancing Expression of Polymerases

To optimize expression of the polymerases of the present invention, inducible or constitutive promoters are well known and may be used to express high levels of a polymerase structural gene in a recombinant host. Similarly, high copy number vectors, well known in the art, may be used to achieve high levels of expression. Vectors having an inducible high copy number may also be useful to enhance expression of the polymerases of the invention in a recombinant host.

To express the desired structural gene in a prokaryotic cell (such as, E. coli B. subtilis, Pseudomonas, etc.), it is necessary to operably link the desired structural gene to a functional prokaryotic promoter. However, the natural promoter of the polymerase gene may function in prokaryotic hosts allowing expression of the polymerase gene. Thus, the natural promoter or other promoters may be used to express the polymerase gene. Such other promoters may be used to enhance expression and may either be constitutive or regulatable (i.e., inducible or derepressible) promoters. Examples of constitutive promoters include the int promoter of bacteriophage λ, and the bla promoter of the β-lactamase gene of pBR322. Examples of inducible prokaryotic promoters include the major right and left promoters of bacteriophage λ (P_{R} and P_{L}), trp, recA, lacZ, lacI, tet, gal, trc, and tac promoters of E. coli. The B. subtilis promoters include a-amylase (Ulmanen et al., J. Bacteriol 162:176-182 (1985)) and Bacillus bacteriophage promoters (Gryczan, T., In: The Molecular Biology Of Bacilli, Academic Press, New York (1982)). Streptomyces promoters are described by Ward et al., Mol. Gen. Genet. 203:468478 (1986)). Prokaryotic promoters are also reviewed by Glick, J. Ind. Microbiol. 1:277-282 (1987); Cenatiempto, Y., Biochimie 68:505-516 (1986); and Gottesman, Ann. Rev. Genet. 18:415-442 (1984). Expression in a prokaryotic cell also requires the presence of a ribosomal binding site upstream of the gene-encoding sequence. Such ribosomal binding sites are disclosed, for example, by Gold et al., Ann. Rev. Microbiol. 35:365404 (1981).

To enhance the expression of polymerases of the invention in a eukaryotic cell, well known eukaryotic promoters and hosts may be used. Preferably, however, enhanced expression of the polymerases is accomplished in a prokaryotic host. The preferred prokaryotic host for overexpressing this enzyme is E. coli.

6. Isolation and Purification of Polymerases

The enzyme(s) of the present invention is preferably produced by fermentation of the recombinant host containing and expressing the desired DNA polymerase gene. However, the DNA polymerases of the present invention may be isolated from any strain which produces the polymerase of the present invention. Fragments of the polymerase are also included in the present invention. Such fragments include proteolytic fragments and fragments having polymerase activity.

Any nutrient that can be assimilated by a host containing the cloned polymerase gene may be added to the culture medium. Optimal culture conditions should be selected case by case according to the strain used and the composition of the culture medium. Antibiotics may also be added to the growth media to insure maintenance of vector DNA containing the desired gene to be expressed. Media formulations have been described in DSM or ATCC Catalogs and Sambrook et al., In: Molecular Cloning, a Laboratory Manual (2nd ed.), Cold Spring Harbor Laboratory Press, Cold Spring Harbor, N.Y. (1989).

Recombinant host cells producing the polymerases of this invention can be separated from liquid culture, for example, by centrifugation. In general, the collected microbial cells are dispersed in a suitable buffer, and then broken down by ultrasonic treatment or by other well known procedures to allow extraction of the enzymes by the buffer solution. After removal of cell debris by ultracentrifugation or centrifugation, the polymerase can be purified by standard protein purification techniques such as extraction, precipitation, chromatography, affinity chromatography, electrophoresis or the like. Assays to detect the presence of the polymerase during purification are well known in the art and can be used during conventional biochemical purification methods to determine the presence of these enzymes.

7. Uses of Polymerases

The polymerases of the present invention may be used in well known nucleic acid synthesis, sequencing, labeling, amplification and cDNA synthesis reactions. Polymerase mutants increased in 3′-5′-exonuclease activity, devoid of or substantially reduced in 5′-3′ exonuclease activity, or containing one or mutations in the O-helix that make the enzyme nondiscriminatory for dNTPs and ddNTPs or containing mutation in the 3′-5′ exonuclease domain which produces an enzyme with reduced misincorporation or increased fidelity, are especially useful for synthesis, sequencing, labeling, amplification and cDNA synthesis. Moreover, polymerases of the invention containing two or more of these properties are also especially useful for synthesis, sequencing, labeling, amplification or cDNA synthesis reactions. As is well known, sequencing reactions (isothermal DNA sequencing and cycle sequencing of DNA) require the use of polymerases. Dideoxy-mediated sequencing involves the use of a chain-termination technique which uses a specific polymer for extension by DNA polymerase, a base-specific chain terminator and the use of polyacrylamide gels to separate the newly synthesized chain-terminated DNA molecules by size so that at least a part of the nucleotide sequence of the original DNA molecule can be determined. Specifically, a DNA molecule is sequenced by using four separate DNA sequence reactions, each of which contains different base-specific terminators (or one reaction if fluorescent terminators are used). For example, the first reaction will contain a G-specific terminator, the second reaction will contain a T-specific terminator, the third reaction will contain an A-specific terminator, and a fourth reaction may contain a C-specific terminator. Preferred terminator nucleotides include dideoxyribonucleoside triphosphates (ddNTPs) such as ddATP, ddTTP, ddGTP, ddITP and ddCTP. Analogs of dideoxyribonucleoside triphosphates may also be used and are well known in the art.

When sequencing a DNA molecule, ddNTPs lack a hydroxyl residue at the 3′ position of the deoxyribose base and thus, although they can be incorporated by DNA polymerases into the growing DNA chain, the absence of the 3′-hydroxy residue prevents formation of the next phosphodiester bond resulting in termination of extension of the DNA molecule. Thus, when a small amount of one ddNTP is included in a sequencing reaction mixture, there is competition between extension of the chain and base-specific termination resulting in a population of synthesized DNA molecules which are shorter in length than the DNA template to be sequenced. By using four different ddNTPs in four separate enzymatic reactions, populations of the synthesized DNA molecules can be separated by size so that at least a part of the nucleotide sequence of the original DNA molecule can be determined. DNA sequencing by dideoxy-nucleotides is well known and is described by Sambrook et al., In: Molecular Cloning, a Laboratory Manual, Cold Spring Harbor Laboratory Press, Cold Spring Harbor, N.Y. (1989). As will be readily recognized, the polymerases of the present invention may be used in such sequencing reactions.

As is well known, detectably labeled nucleotides are typically included in sequencing reactions. Any number of labeled nucleotides can be used in sequencing (or labeling) reactions, including, but not limited to, radioactive isotopes, fluorescent labels, chemiluminescent labels, bioluminescent labels, and enzyme labels. For example the polymerases of the present invention may be useful for incorporating αS nucleotides ([αS]dATP, [αS]dTTP, [αS]dCTP and [αS]dGTP) during sequencing (or labeling) reactions.

Polymerase chain reaction (PCR), a well known DNA amplification technique, is a process by which DNA polymerase and deoxyribonucleoside triphosphates are used to amplify a target DNA template. In such PCR reactions, two primers, one complementary to the 3′ termini (or near the 3′-termini) of the first strand of the DNA molecule to be amplified, and a second primer complementary to the 3′ termini (or near the 3′-termini) of the second strand of the DNA molecule to be amplified, are hybridized to their respective DNA strands. After hybridization, DNA polymerase, in the presence of deoxyribonucleoside triphosphates, allows the synthesis of a third DNA molecule complementary to all or a portion of the first strand and a fourth DNA molecule complementary to all or a portion of the second strand of the DNA molecule to be amplified. This synthesis results in two double stranded DNA molecules. Such double stranded DNA molecules may then be used as DNA templates for synthesis of additional DNA molecules by providing a DNA polymerase, primers, and deoxyribonucleoside triphosphates. As is well known, the additional synthesis is carried out by “cycling” the original reaction (with excess primers and deoxyribonucleoside triphosphates) allowing multiple denaturing and synthesis steps. Typically, denaturing of double stranded DNA molecules to form single stranded DNA templates is accomplished by high temperatures. The DNA polymerases of the present invention are preferably heat stable DNA polymerases, and thus will survive such thermal cycling during DNA amplification reactions. Thus, the DNA polymerases of the invention are ideally suited for PCR reactions, particularly where high temperatures are used to denature the DNA molecules during amplification.

8. Kits

A kit for sequencing DNA may comprise a number of container means. A first container means may, for example, comprise a substantially purified sample of the polymerases of the invention. A second container means may comprise one or a number of types of nucleotides needed to synthesize a DNA molecule complementary to DNA template. A third container means may comprise one or a number of different types of terminators (such as dideoxynucleoside triphosphates). A fourth container means may comprise pyrophosphatase. In addition to the above container means, additional container means may be included in the kit which comprise one or a number of primers and/or a suitable sequencing buffer.

A kit used for amplifying or synthesis of nucleic acids will comprise, for example, a first container means comprising a substantially pure polymerase of the invention and one or a number of additional container means which comprise a single type of nucleotide or mixtures of nucleotides.

Various primers may be included in a kit as well as a suitable amplification or synthesis buffers.

When desired, the kit of the present invention may also include container means which comprise detectably labeled nucleotides which may be used during the synthesis or sequencing of a nucleic acid molecule. One of a number of labels may be used to detect such nucleotides. Illustrative labels include, but are not limited to, radioactive isotopes, fluorescent labels, chemiluminescent labels, bioluminescent labels and enzyme labels.

Having now generally described the invention, the same will be more readily understood through reference to the following Examples which are provided by way of illustration, and are not intended to be limiting of the present invention, unless specified.

### EXAMPLE 1

### Construction of Hybrid Taq DNA Polymerase

All three domains of Taq polymerase have been described by Kim et. al. (Nature 376: 612-616 (1995)) from the crystal structure. The active 5′-3′-exonuclease domain resides within 1-289 amino acids, the inactive 3′-5′-exonuclease domain resides within 294-422 amino acids and the active polymerase domain resides within 424-831 amino acids. From the amino acids alignment between Taq and Tne DNA polymerase, we estimated that the corresponding regions for Tne polymerase are as follows: 1-291 amino acids (5′-3′-exonuclease), 292-485 amino acids (3′-5′-exonuclease) and 486-893 amino acids (polymerase). First, we wanted to replace the 5′-3′-exonuclease domain from Tne DNA polymerase with the 5′-3′-exonuclease domain of Taq polymerase. Since there was a convenient BsrGI within the 5′-3-exonuclease domain (amino acids 204-206) of Tne polymerase, we have utilized this site for domain swapping. 5′-3′-exnuclease domain of Taq polymerase was amplified with the following oligos:

5′-ATTATTGAGCTCTAAGGAGATATCATATGCGCGGCATGCTG (oligo #1; SEQ ID NO:1)

5′-AATAATAAG CTGTACAGCCGTCTTCTCCCCGATGCC (oligo #2; SEQ ID NO:2)

The oligo #1 contains two restriction sites, SstI (bold underlined) and NdeI (bold italics) and the oligo #2 contains a BsrGI site for ease of cloning the PCR fragment. The PCR Supermix (Invitrogen Corporation, Life Technologies Division) was used for amplification with the concentration of each primer being 1 uM. A PCR program of 94° for 2 min (1 cycle), 94° C for 15 sec, 55° C. for 15 sec, 72° C. for 45 sec (15 cycles); 72° C. for 2 min (1 cycle) was used in a Perkin Elmer thermocycler. The PCR product was digested with SstI and BsrGI and cloned into pTTQTne (PTTQ, Pharmacia, California). The plasmid was designated as pTne79. This plasmid contains a mutation to inactivate the 3′-5′-exonuclease activity. The BsrGI-HindIII fragment of pTne79 was replaced with the identical fragment from wild-type Tne polymerase gene to restore the 3′-5′-exonuclease activity. This plasmid is called pTne8O. This clone contains 5′-3′-exonuclease domain of Taq polymerase and the active 3′-5′exonuclease and polymerase domains from Tne polymerase. To replace the polymerase domain from pTne8O, we replaced amino acids 515-893 of Tne polymerase with amino acids 454-831 of Taq polymerase. The Taq polymerase domain was amplified using the following oligos:

5′ GTGCGCCTGGACGTGGAATCCCTCCGGGCCTTGTCCCTG (oligo# 3; SEQ ID NO:3)

5′ ATATATTAAGCTT CACTCCTTGGCGGAGAGCCAGTC (oligo # 4;

SEQ ID NO:4)

In the oligo # 3, an EcoRI site was created and in the oligo # 4, a HindIII site was created so that the PCR product could be cloned to replace the EcoRI-HinDIII fragment of pTne 80. There are two EcoRI sites in Tne polymerase domain (within amino acids 516-517 and 621-622, respectively). The HindIII site is outside the polymerase gene and present in the vector. The PCR was done as described above. The PCR product was digested with EcoRI and HindIII and cloned into EcoRI+HindIII digested pTne 80. This plasmid was called pTne 86. It contains the ⁵′-³′-exonuclease and the polymerase domains from Taq polymerase and the ³′-5′-exnuclease domain from Tne polymerase. In the oligo #3, the codon CGG for arginine was used instead of AGG in Taq polymerase (amino acid 457). In this construct, the junction at the polymerase domain is between β-sheet 6 and helix H. Another hybrid was made at a different location. (See Example 4).

The sequence at the 3′-5′-exonuclease and the polymerase domain junctions is as follows:

----KGIGEKTA²⁰⁴V²⁰⁵QLLG---------GVYVDTEF⁵¹⁷L⁴⁵⁶RALS
LEV----(SEQ ID NO:5)
BsrGI EcoRI

The bold italics sequences are derived from Taq polymerase and the others are from Tne polymerase. The numbers correspond to the amino acid number of each respective polymerase.

### EXAMPLE 2

### Preliminary Screening of Hybrids for Polymerase Activity

The constructs were analyzed for thermostable polymerase activity as follows: Overnight cultures were grown (2ml) in Circle Grow (CG) containing ampicillin (100 ug/ml) at 30° C. To 40 ml of CG +Amp¹⁰⁰, 1 ml of the overnight culture was added and the culture was grown at 37° C. until it reached an O.D of about 1.0 (A₅₉₀). The culture was split into two 20 ml aliquots, and the first aliquot (uninduced) was kept at 37° C. To the other aliquot, IPTG was added to a final concentration of 2 mM and the culture was incubated at 37° C. After 3 hours, the cultures were spun down at 4° C. in a table-top centrifuge at 3500 rpm for 20 minutes. The supernatant was poured off and the cell pellet was stored at −70° C. The cell pellet was suspended in Iml of buffer containing 10 mM Tris pH 8.0, 1 mM Na₂EDTA, 10 mM β-mercaptoethanol (β-ME). The cell suspension (500 ul) was heated at 74° C. for 20 minutes in a water bath. The tubes were kept on ice for 10 minutes and then centrifuged at 13000 rpm for 10 min at 4° C. The clear supernatant was removed assayed for polymerase activity at 72° C. The polymerase activity assay reaction mixture contained 25 mM TAPS buffer (pH 9.3), 2 mM MgCl₂, 15 mM KCl, 1 mM EDTA, 0.2 mM dNTPs, 500 ug/ml DNAseI-treated salmon sperm DNA, 21 mCi/ml α³²PdCTP, and various amounts of enzyme as specified in each example in a final volume of 25 ul. After 10 min incubation at 72° C., 5 ul of 0.5 M EDTA was added to the tube. TCA precipitable counts were measured in GF/C filters using 25 ul of reaction mixture.

### EXAMPLE 3

### Purification of Hybrid Polymerase from pTne 86

The cells were grown in Circle Grow (Bio 101, California) at 30° C. and induced with 1 mM IPTG. Two to three grams of cells expressing cloned mutant Tne DNA polymerase were resuspended in 15-20 ml of sonication buffer (50 mM Tris-HCl, pH 8.0, 10% glycerol, 5 mM β-Me, 50 mM NaCl, 1 mM EDTA and 0.5 mM PMSF) and sonicated with a 550 Sonic Dismembrator. The sonicated sample was heated at 75° C. for 30 min. A solution of sodium chloride was added to the sample to increase the concentration to 200 mM and solution of 5% PEI (polyethylimine) was added dropwise to a final concentration of 0.2%. The sample was centrifuged at 13,000 rpm for 10 min. Ammonium sulfate (305 mg/ml) was added to the supernatant. The pellet was collected by centrifugation and resuspended in 5 ml of MonoQ column buffer (5OmM Tris-HCl pH 8.0, 10% glycerol, 5mM β-ME, 50 mM NaCl and 1 mM EDTA). The sample was dialyzed against 250 ml of MonoQ buffer overnight. Following centrifugation of the sample at 13,000 rpm to remove any insoluble materials, it was loaded onto a MonoQ column (HR5/5, Pharmacia). The column was washed with MonoQ column buffer to a baseline of A280 and then eluted with a 20 column volume linear gradient of 50-300 mM NaCl in MonoQ column buffer. The fractions were analyzed by SDS-PAGE and were assayed for thermostable polymerase activity as described above.

### EXAMPLE 4

### Hybrid Taq Polymerase from a New Junction at the Polymerase Domain

In this case, the junction is created between Helix F and Helix G. A ClaI site is created to connect the two domains. The oligos for PCR were the following:

| | |
|---|---|
| 5′ AAG ACG GCT GTA CAG CTT CTC GGC AAG | (oligo # 5; SEQ ID NO:6) |

This oligo anneals to the amino end of the Tne 3′-5′-exonuclease domain.

| | |
|---|---|
| 5′ GAG CTT CAT CGA TAG TAT CTT GTA GAG CCT ATA AGT | (oligo # 6; SEQ ID NO:7) |

This oligo anneals to the carboxyl end of the Tne 3′ exo domain.

| | |
|---|---|
| 5′ ATA CTA TCG ATG AAG CTC CAT GAA GAG AGG CTC CTT TGG | (oligo #7; SEQ ID NO:8) |
| CTT TAC CGG GAG | |

This oligo anneals at the amino end of the Taq polymerase domain.

The restriction enzyme sites in the oligos are in bold italics. The oligo #5 contains a BsrGI site and oligo # 6 and # 7 contains Clal site. PCR Supermix (Invitrogen Corporation, Life Technologies Division, Rockville, Md.) was used for amplification with the concentration of each primer being 1 uM. A PCR program of 94° for 2 min; 94° C. for 15 s, 55° C. for 15 s, 72° C. for 45 s, (15 times); 72° C. for 2 min was used in a Perkin Elmer (California) thermocycler. Amplification with oligos #5 and #6 using Tne DNA polymerase gene as the template gives the 850 bp product and amplification with oligos #7 and #4 using Taq DNA polymerase gene as the template gives a 1300 bp PCR product. These were digested with the restriction enzymes BsrGI/ClaI and ClaI/HindIII, respectively. The vector pTne 86 was digested with BsrGI/HindlIl and the three fragments were ligated using T4 DNA Ligase. The clones were analyzed by restriction enzyme analysis. The clone is designated as pTne 173 and produces active polymerase as described above.

The sequence at the 3′-5′-exonuclease domain junction is similar to pTne 86. The sequence at the polymerase junction is as follows:

| | |
|---|---|
| L S M K L H E⁴⁸⁵E⁴²⁴R L L W L Y | (SEQ ID NO:9) |

We have made other hybrids using the similar technique with different junction at the polymerase domain keeping the 3′-5′-exonuclease junction similar to pTne 86. The sequences at the polymerase junction of several constructs are as follows:

| | | |
|---|---|---|
| pTne 87: | ------L S M⁴⁸¹ R 419 L E G E E R L L-------------- | (SEQ ID NO:10) |
| pTne90: -----R I H A S⁶²⁵ F⁵⁶⁴ N Q T A T------------------ | (SEQ ID NO:11) | |

Both pTne 87 and pTne 90 produce active polymerase as assayed above.

### EXAMPLE 5

### 3′-5′ Exonuclease Activity Assay of Hybrid Taq Polymerase

The purified hybrid polymerase from pTne 86 was studied in detail for catalytic activities. The editing function (3′-5′exonuclease activity) of the engineered polymerase was qualitatively measured using a double stranded DNA, 36/60 primer/template, having 4 mismatch base pairing at the 3′-termini of the primer. The 3′-5′ exonuclease activity of the wild-type Taq polymerase and the chimeric enzyme were assayed at 60° C. For control, the efficiency of the 3′-5′ exonuclease activity of two Tne polymerase mutants was also assayed. The first mutant derivative was deficient in the 5′-3′ exonuclease activity due to the mutation at D1 37A and the second was deficient in both the 3′-5′ and 5′-3′ exonuclease activities due to the double substitution at D323A and D137A, respectively.

The following DNA substrate with a four-base mismatch was used for the assay:

5′-GCTCCGCGACGGCAGCCACGGCGTCGGCCGGCGGTT-3′ (SEQ ID NO:12)

3 ′-CGAGGCGCTGCCGTCGGTGCCGCAGCCGGCCGGTTTCTGCTAC GCCGGTAGGCTAACGTTACG-5′ (SEQ ID NO:13)

Degradation of the 3′-termini of the primer strand was initiated by the addition of the polymerase in the presence of MgCl₂. The reaction mixture contained approximately 20 nM DNA in 20 mM Tris-HCl, pH 8.4, 1.5 mM MgCl₂ and 50 mM KCl. The polymerases were in significant excess compared to the DNA substrate so as to catalyze the cleavage of the phosphodiester bonds under pre-steady state conditions. The reaction was quenched at 20 sec, 1 min. and 2 min following the addition of the polymerase by removing 1.5 ul of samples and mixed with 3 ul of a stop solution containing formamide, EDTA, SDS, bromophenol blue and Xylene cyanol FF. Finally, the samples were fractionated on a denaturing 8% polyacrylamide gel.

DNA Substrate Preparation

The oligonucleotides (primer and template strands) were ordered from Custom Primers, Invitrogen Corporation, Life Technologies Division. The primer strand was HPLC purified, whereas the template strand was PAGE purified. The primer was 5′-labeled using T4 polynucleotide kinase and was annealed to the template.

Result

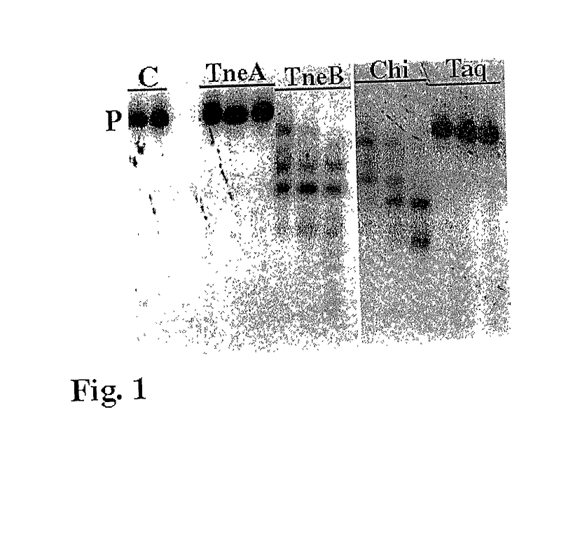

The chimeric polymerase degrades the mismatch bases with about similar efficiency as Tne polymerase under our experimental condition (FIG. 1). As expected, the wild-type Taq and the Tne (3′-5′ exonuclease minus mutant) polymerases did not catalyze the cleavage of primer. This result indicates that the chimeric polymerase was enzymatically active suggesting a Taq polymerase that is capable of editing mismatches.

### EXAMPLE 6

### Quantitative 3′-5′ Exonuclease Activity Assay

The 3′-5′ exonuclease activity of wild type Taq DNA polymerase, Tne DNA polymerase (5′-exo⁻, 3′-5′-exo⁺) and Taq/Tne hybrid DNA polymerase was measured using a 3′-labeled double stranded DNA. The substrate used was Taq I restriction enzyme digested lambda DNA fragments labeled at the 3′-end with ³HdGTP and ³HdCTP in the presence of E. coli DNA polymerase I. One pmol of the substrate was used in 50 ul reaction containing 20 mM Tris-HCl, pH 8.0, 50 mM KCl, 2 mM MgCl₂, 5 mM dithiothreotol (DTT) with approximately 2.5 units of different polymerases. In the case of wild type Taq DNA polymerase, approximately 21 units were also included. The reaction was incubated for 1 hr at 72° C. The tubes were placed on ice and 10 ul of each reaction was spotted on a PEI plate. Thin layer chromatography was carried out in 2 N HCl. Release of terminal label was measured by liquid scintillation.

Result: As expected, negligible amount of labeled nucleotide was released by 3′-5′-exonuclease mutant of Tne polymerase and wild type Taq polymerase with either 2.6 or 21 units of enzyme (Table 2). However, both 3′-5′-exonuclease proficient Tne polymerase and the Taq/Tne hybrid DNA polymerase (or Taq hybrid produced from pTne 86) released almost equal amount of labeled nucleotide. This is apparent that full 3′-5′ exonuclease activity of Tne polymerase activity has been recovered in the hybrid polymerase. The increase of 3′-5′ exonuclease activity in the hybrid polymerase was estimated to be 40 fold compared to the wild type Taq polymerase.

TABLE 2
Exonuclease assay on 3′ ds DNA substrate

| | | | | | % | |
|---|---|---|---|---|---|---|
| | | ug | % | % | releasing/ | Relative |
| Enzyme | Units | Protein | released | released/U | ug | activity |
| Taq wt | 2.60 | 0.15 | 3.8 | 1.46 | 25.3 | 1 |
| | 21.0 | 1.2 | 4.6 | 0.2 | 3.8 | — |
| Tne (3′exo⁻⁾ | 2.75 | 0.1 | 4.2 | 4.2 | 42.0 | 1.7 |
| Tne (3′exo⁺⁾ | 2.60 | 0.08 | 74.0 | 28.5 | 925.0 | 3.0 |
| Taq hybrid | 2.40 | 0.06 | 72.0 | 30.0 | 1200.0 | 48 |

### EXAMPLE 7

### Coupled Polymerase/Exonuclease Activity Determination

We designed an experiment in order to investigate the ability of the hybrid polymerase to degrade mismatch primer termini and concurrently elongate the primer that is annealed to a complementary template. The exonuclease directed degradation of the primer followed by the polymerization reaction was assayed using the above DNA substrate under similar conditions described above. The exception is the presence of dNTP in this reaction mixture, in order to elongate the primer. The final concentration of dNTP was 250 uM.

Result

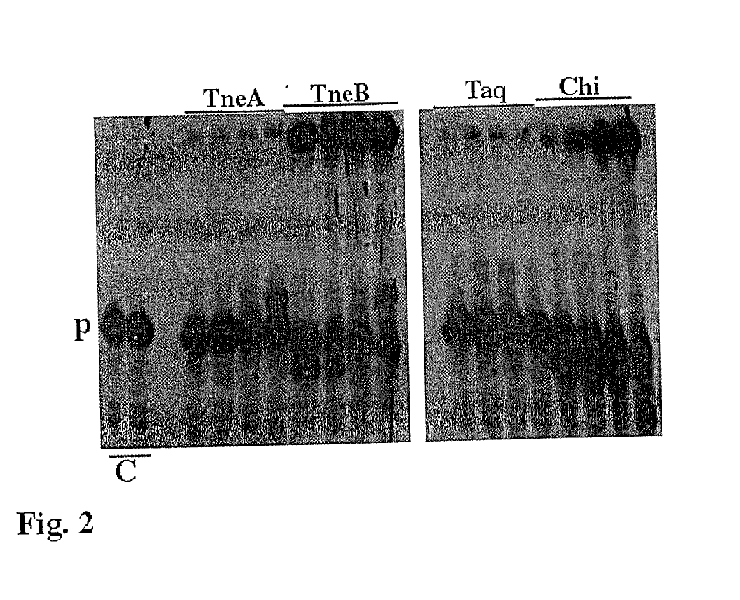

The chimeric polymerase degrades the mismatch bases of the primer 3′-termini and elongates it with about similar efficiency as Tne polymerase under our experimental condition (FIG. 2). As expected, the wild-type Taq (lacking 3′-5′ exonuclease activity) and the Tne (3′-5′ exonuclease minus mutant) polymerases did not cleave at the 3′-termini of primer. This result also indicates that the chimeric polymerase is enzymatically active suggesting that it has folded correctly.

### EXAMPLE 8

### Steady State Kcat Determination

The steady state Kcat for the chimeric DNA polymerase was determined as described by Polesky et al., 1990 at 60° C. The DNA substrate was prepared by annealing (dG)₃₅ to poly(dC) at a molar ratio of about 1 (dG)₃₅ per 100 template G residues. The concentration of DNA and dNTP at which the rate was determined were 2.5 uM and 250 uM dNTP, respectively.

Result

The steady state k(cat) for the chimeric DNA polymerase to incorporate dGTP is about 25 sec⁻¹. This result is the same to the value derived for Tne and Taq DNA polymerases suggesting that the chimeric DNA polymerase has folded similar to the native structures of the parent proteins.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A polymerase which has been modified or mutated to increase or enhance fidelity.

2. A polymerase which has been modified or mutated to reduce or eliminate misincorporation of nucleotides during nucleic acid synthesis.

3. The polymerase of claim 1 or 2, wherein said polymerase is a DNA polymerase.

4. The polymerase of claim 3, wherein said polymerase is mesophilic or thermostable.

5. The polymerase of claim 3, wherein said polymerase is selected from the group consisting of Tne DNA polymerase, Taq DNA polymerase, Tma DNA polymerase, Tth DNA polymerase, Tli, VENT™ DNA polymerase, Pfu DNA polymerase, DEEPVENT™, DNA polymerase, Pwo DNA polymerase, Bst DNA polymerase, Bca DNA polymerase, Tfl DNA polymerase, and mutants, variants and derivatives thereof.

6. The polymerase of claim 1 or 2 which further comprises one or more modifications or mutations that reduce or eliminate an activity selected from the group consisting of:
(a) the 5 ′-3′ exonuclease activity of the polymerase; and,
(b) the discriminatory activity against one or more dideoxynucleotides.

7. The polymerase of claim 6, which is modified or mutated to increase 3′-5′ exonuclease activity.

8. The polymerase of claim 6, which is modified or mutated to reduce or eliminate discriminatory activity.

9. The polymerase of claim 6, which is modified or mutated to reduce or eliminate 5′-3′ exonuclease activity.

10. The polymerase of claim 3, which comprises one or more mutations or modifications in the 3′-5′ domain of said polymerase.

11. The polymerase of claim 10, wherein said mutation or modification is a substitution of the 3′-5′-exonuclease domain with a 3′-5′-exonuclease domain having increased 3′-5′-exonuclease activity.

12. The polymerase of claim 11, wherein said polymerase is Taq.

13. The polymerase of claim 12, wherein said 3′-5′ exonuclease domain having increased activity is from Tne polymerase.

14. A vector comprising a gene encoding the polymerase of any one of claims 1 and 2.

15. The vector of claim 14, wherein said gene is operably linked to a promoter.

16. The vector of claim 15, wherein said promoter is selected from the group consisting of a λ-P_{L} promoter, a tac promoter, a trp promoter, and a trc promoter.

17. A host cell comprising the vector of claim 14.

18. A method of producing a polymerase, said method comprising:
(a) culturing the host cell of claim 17;
(b) expressing said gene; and
(c) isolating said polymerase from said host cell.

19. The method of claim 18, wherein said host cell is E. coli.

20. A method of synthesizing a nucleic acid molecule comprising:
(a) mixing a nucleic acid template with one or more polymerases of claim 1 or 2; and
(b) incubating said mixture under conditions sufficient to make a nucleic acid molecule complementary to all or a portion of said template.

21. The method of claim 20, wherein said mixture further comprises one or more nucleotides selected from the group consisting of dATP, dCTP, dGTP, dTTP, dITP, 7-deaza-dGTP, dUTP, ddATP, ddCTP, ddGTP, ddlTP, ddTTP, [α-S]dATP, [α-S]dTTP, [α-S]dGTP, and [α-S]dCTP.

22. The method of claim 21, wherein one or more of said nucleotides are detectably labeled.

23. A method of sequencing a DNA molecule, comprising:
(a) hybridizing a primer to a first DNA molecule;
(b) contacting said DNA molecule of step (a) with deoxyribonucleoside triphosphates, the DNA polymerase of any one of claims 1 or 2, and a terminator nucleotide;
(c) incubating the mixture of step (b) under conditions sufficient to synthesize a random population of DNA molecules complementary to said first DNA molecule, wherein said synthesized DNA molecules are shorter in length than said first DNA molecule and wherein said synthesized DNA molecules comprise a terminator nucleotide at their 5′ termini; and
(d) separating said synthesized DNA molecules by size so that at least a part of the nucleotide sequence of said first DNA molecule can be determined.

24. The method of claim 23, wherein said deoxyribonucleoside triphosphates are selected from the group consisting of dATP, dCTP, dGTP, dTTP, dITP, 7-deaza-dGTP, dUTP, [α-S]dATP, [α-S]dTTP, [α-S]dGTP, and [α-S]dCTP.

25. The method of claim 23, wherein said terminator nucleotide is ddTTP, ddATP, ddGTP, ddITP or ddCTP.

26. The method of claim 23, wherein one or more of said deoxyribonucleoside triphosphates is detectably labeled.

27. The method of claim 23, wherein one or more of said terminator nucleotides is detectably labeled.

28. A method for amplifying a double stranded DNA molecule, comprising:
(a) providing a first and second primer, wherein said first primer is complementary to a sequence within or at or near the 3′-termini of the first strand of said DNA molecule and said second primer is complementary to a sequence within or at or near the 3′-termini of the second strand of said DNA molecule;
(b) hybridizing said first primer to said first strand and said second primer to said second strand in the presence of the DNA polymerase of any one of claims 1 or 2, under conditions such that a third DNA molecule complementary to all or a portion of said first strand and a fourth DNA molecule complementary to all or a portion of said second strand are synthesized;
(c) denaturing said first and third strand, and said second and fourth strands; and
(d) repeating steps (a) to (c) one or more times.

29. The method of claim 28, wherein said conditions comprise the presence of deoxyribonucleoside triphosphates selected from the group consisting of dATP, dCTP, dGTP, dTTP, dITP, 7-deaza-dGTP, dUTP, [α-S]dATP, [α-S]dTTP, [α-S]dGTP, and [α-S]dCTP.

30. A kit for sequencing a DNA molecule comprising one or more polymerases of any one of claims 1 or 2.

31. The kit of claim 30 further comprising one or more dideoxyribonucleoside triphosphates and/or one or more deoxyribonucleoside triphosphates.

32. A kit for amplifying or synthesizing a nucleic acid molecule comprising one or more polymerases of any one of claims 1 and 2.

33. The kit of claim 32, further comprising one or more deoxyribonucleoside triphosphates.

34. A method of preparing cDNA from mRNA, comprising
(a) mixing one or more mRNA templates with one or more polymerases of claim 1 or 2; and
(b) incubating said mixture under conditions sufficient to synthesize a cDNA molecule complementary to all or a portion of said templates.

35. The method of claim 34, further comprising incubating said synthesized cDNA under condition to make double stranded cDNA.
